(19) United States
(12) Patent Application Publication
(10) Pub. No.: US 2026/0315067 A1
(43) Pub. Date: Oct. 15, 2026

(54) TISSUE SUTURING DEVICE AND OPERATING METHOD THEREOF AND OPERATING ASSEMBLY

(71) Applicant: Cilag GmbH International, Zug (CH)

(72) Inventors: Sijin HE, Shanghai (CN); Jie ZHANG, Shanghai (CN); Shaohua BU, Shanghai (CN)

(21) Appl. No.: 19/486,051

(22) PCT Filed: May 24, 2024
(86) PCT No.: PCT/EP2024/064334

§ 371 Date: Nov. 20, 2025

(30) Foreign Application Priority Data
May 24, 2023 (CN) 202310594832.X

(51) Int. Cl. A61B 17/04; A61B 17/06; A61B 17/062

(52) U.S. Cl. CPC A61B 17/0491; A61B 17/0482; A61B 17/06061; A61B 17/062

(57) ABSTRACT

The present invention relates to the technical field of surgical instruments, and provides a tissue suturing device and an operating method thereof and an operating assembly. In the operating assembly, an operating member includes a wing driving path, a transition path, and a needle driving path which are sequentially implemented. When moving along the wing driving path to the transition path, the operating member is not engaged with a needle mounting member so that the needle mounting member is allowed to be maintained in a first position, the operating member is engaged with a wing driving member so that the wing driving member is driven to move from a third position to a fourth position, and the wing driving member can be maintained in the fourth position; when moving along the transition path to the needle driving path, the operating member is disengaged from the wing driving member and engaged with the needle mounting member; and when moving along the needle driving path, the operating member drives the needle mounting member to move from the first position to a second position. The operating assembly sequentially operates stabilizing wings and suturing needles, thereby avoiding accidental extension of the suturing needles, and significantly improving the safety of using the tissue suturing device.

### TECHNICAL FIELD

The present invention relates to the technical field of surgical instruments, and specifically relates to an operating assembly for a tissue suturing device, a tissue suturing device, and an operating method of a tissue suturing device.

### BACKGROUND OF THE INVENTION

During an operation, surgeons usually need to use tissue closure instruments to suture a patient's wound to be sutured, such as an incision of the abdominal cavity.

Existing tissue closure instruments typically include a cannula, stabilizing wings, and a puncture needle. The stabilizing wings are arranged at a distal end of the cannula, and the puncture needle is arranged within the cannula, and can extend out of the cannula. When in use, an operator inserts the cannula into the wound to be sutured, and then operates the tissue closure instrument to unfold the stabilizing wings on the inside surface of the tissue of the wound to be sutured, thereby stabilizing the cannula. Then, the puncture needle is operated to extend out of the cannula and pass through the tissue to reach the stabilizing wings, thereby completing the arrangement of the suture.

During actual suturing, a skilled surgeon can achieve correct puncture of the puncture needle by virtue of experience. For many surgeons, however, due to various factors such as inexperience or accidental contact, the suturing needle will usually be extended out of the cannula before the stabilizing wings are unfolded, which may cause unnecessary injury to patients or the surgeons themselves.

### BRIEF SUMMARY

In view of the above problems existing in the prior art, a first aspect of the present invention provides an operating assembly for a tissue suturing device. The operating assembly can sequentially operate the stabilizing wings and the suturing needle, and avoid accidental extension of the suturing needle, thereby significantly improving the safety of using the tissue suturing device.

In order to achieve the objective described above, the present invention provides an operating assembly for a tissue suturing device. The operating assembly comprises a tube body, a needle mounting member, a wing driving member, and an operating member, wherein the tube body comprises a proximal end, a distal end, and an axial channel extending between the proximal end and the distal end; the needle mounting member is axially and movably arranged in the axial channel; the wing driving member is axially and movably arranged in the axial channel; at least a part of the operating member is movably arranged in the axial channel; wherein the operating member comprises a wing driving path, a transition path, and a needle driving path which are sequentially implemented; wherein when moving along the wing driving path from an initial position to the transition path, the operating member is not engaged with the needle mounting member so that the needle mounting member is allowed to be maintained in a first position used to enable suturing needles to be in a retracted state, the operating member is engaged with the wing driving member so that the wing driving member is driven to move from a third position used to enable stabilizing wings to be in a folded state to a fourth position used to enable the stabilizing wings to be in an unfolded state, and the wing driving member is capable of being maintained in the fourth position to maintain the stabilizing wings in the unfolded state; when moving along the transition path to the needle driving path, the operating member is disengaged from the wing driving member and engaged with the needle mounting member; and when moving along the needle driving path, the operating member drives the needle mounting member to move from the first position to a second position used to enable the suturing needles to be in an extended state.

In this technical solution, both the driving of the needle mounting member and the driving of the wing driving member are implemented by means of the operating member, and the operating member comprises the wing driving path, the transition path, and the needle driving path which are sequentially implemented. Hence, during actual use, in the wing driving path, the operating member can only drive the wing driving member but cannot drive the needle mounting member. In this case, even if the operating member is incorrectly operated or accidentally touched, the operating member cannot drive the needle mounting member to move axially, and the suturing needles mounted on the needle mounting member thus cannot extend out of the tube body. In this way, accidental extension of the suturing needles can be completely avoided, thereby significantly improving the safety of using the tissue suturing device. By means of the transition path, the operating member is disengaged from the wing driving member and engaged with the needle mounting member. In this way, in the needle driving path, the operating member can drive the needle mounting member to move from the first position to the second position, such that the suturing needles extend out of the tube body to a puncture position.

In some embodiments, the wing driving path, the transition path, and the needle driving path are formed between the tube body and the operating member, wherein the wing driving path extends toward the proximal end and along the axial direction, the needle driving path extends toward the distal end and along the axial direction, and the transition path is arranged along a circumferential direction of the tube body, such that the operating member is capable of moving axially along the wing driving path, then rotating along the transition path by a preset angle and then reaching the needle driving path, and then moving axially along the needle driving path.

In some embodiments, a wing driving guide slot providing the wing driving path, a transition guide slot providing the transition path, and a needle driving guide slot providing the needle driving path are formed on an axial sidewall of the tube body, wherein a guide column is provided on the operating member and is movable along the wing driving guide slot, the transition guide slot, and the needle driving guide slot.

In some embodiments, the tube body comprises two half tubes capable of being joined together, wherein the wing driving path, the transition path, and the needle driving path are formed between at least one of the two half tubes and the operating member.

In some embodiments, each of the half tubes is formed with a wing driving guide slot, a transition guide slot, and a needle driving guide slot, two guide columns are provided on the operating member, and each of the guide columns is movable along the wing driving guide slot, the transition guide slot, and the needle driving guide slot of a half tube corresponding thereto.

In some embodiments, when the operating member moves along the transition path to the needle driving path, the wing driving member maintained in the fourth position is capable of providing guidance for movement of the operating member along the needle driving path.

In some embodiments, an axially extending mating slot is formed on an end surface of one of the operating member and the wing driving member, and an axially extending mating section is formed at an end part of the other of the operating member and the wing driving member, wherein when the operating member moves axially along the wing driving path, the mating section is offset from the mating slot, such that the operating member is engaged with the wing driving member; when the operating member rotates along the transition path to the needle driving path, the mating section is axially aligned with the mating slot, such that the operating member is disengaged from the wing driving member; and when the operating member moves axially along the needle driving path, the mating section enters the mating slot to provide the guidance.

In some embodiments, the needle mounting member is formed with an axial through-hole, and an axial open slot and a circumferential open slot communicating with each other are formed on a sidewall of the axial through-hole; an extension section capable of extending into the axial through-hole is formed at a distal end of the operating member, and a guide pins radially extending outwards is arranged on the extension section; wherein the operating member, when moving axially along the wing driving path towards the distal end, drives the guide pin to slide axially along the axial open slot, such that the operating member, when not driving the needle mounting member to move axially towards the distal end, enables the needle mounting member to be maintained in the first position; and the operating member, when rotating circumferentially along the transition path, drives the guide pin to slide circumferentially along the circumferential open slot, such that the operating member is engaged with the needle mounting member.

In some embodiments, at least two sets of the axial open slot and the circumferential open slot are formed on the sidewall of the axial through-hole, and at least two guide pins are provided on the extension section, wherein each of the guide pins is capable of sliding along a set of the axial open slot and the circumferential open slot corresponding thereto.

In some embodiments, a reference mark is arranged on the tube body, a wing driving mark and a needle driving mark circumferentially spaced apart at a preset angle and axially spaced apart from each other are arranged on the operating member, and the wing driving mark is different from the needle driving mark, wherein when the wing driving mark is axially aligned with the reference mark, the operating member is capable of moving axially along the wing driving path from the initial position; and when the operating member rotates along the transition path by the preset angle to reach the needle driving path, the needle driving mark is axially aligned with the reference mark.

In some embodiments, the needle mounting member is formed with a central through-hole, and the wing driving member passes through the central through-hole.

In some embodiments, the wing driving member is provided with a positioning member, and a releasable locking structure is formed between the positioning member and the tube body, wherein when the operating member moves along the wing driving path from the initial position to the transition path, the locking structure is in a locking state to maintain the wing driving member in the fourth position; and when the locking structure is released, the wing driving member is allowed to move from the fourth position to the third position.

In some embodiments, an operating opening and a locking opening axially spaced apart from each other are formed on a sidewall of the tube body, the positioning member comprises an elastic arm, the elastic arm is provided with a pressing part and a locking hook axially spaced apart from each other, and the pressing part protrudes outwards radially from the operating opening; the locking structure comprises the locking opening and the locking hook, and is in the locking state when the locking hook is in the locking opening; and the elastic arm is pressed by means of the pressing part, such that the locking structure is released when the locking hook is disengaged from the locking opening.

In some embodiments, the positioning member is formed with suturing needle channels used for allowing the suturing needles to pass through axially; and/or, axial recessed portions allowing the suturing needles to pass through are formed on the elastic arm of the positioning member.

In some embodiments, when the needle mounting member is in the second position, the positioning member stops the needle mounting member.

In some embodiments, an accommodating slot is formed on a proximal end surface of the positioning member, a stop column is formed at a distal end of the needle mounting member, and when the needle mounting member is in the second position, the stop column extends into the accommodating slot.

In a second aspect, the present invention provides a tissue suturing device. The tissue suturing device comprises the operating assembly according to any of the foregoing described in the first aspect above, stabilizing wings, and suturing needles, wherein the stabilizing wings are hinged to the distal end of the tube body and hinged to the wing driving member, and the suturing needles are arranged on the needle mounting member and located in the tube body, wherein the stabilizing wings are capable of being unfolded and folded, and the suturing needles are capable of extending out of the tube body to a puncture position and retracting back into the tube body.

In this way, as described in the first aspect above, by means of the operating assembly, the tissue suturing device can completely avoid accidental extension of the suturing needle, thereby significantly improving the safety of using the tissue suturing device.

In a third aspect, the present invention provides an operating method of a tissue suturing device. The operating method comprises a wing driving path, a transition path, and a needle driving path which are sequentially implemented, wherein an operating member of a tissue suturing device is driven to move along the wing driving path from an initial position to the transition path, such that the operating member only drives a wing driving member of the tissue suturing device to move from a third position to a fourth position and be maintained in the fourth position, so as to unfold stabilizing wings of the tissue suturing device; the operating member is driven to move along the transition path to the needle driving path, such that the operating member is disengaged from the wing driving member and engaged with a needle mounting member of the tissue suturing device; and the operating member is driven to move along the needle driving path to drive the needle mounting member to move from a first position to a second position, such that suturing needles are driven to extend out of a tube body of the tissue suturing device to a puncture position.

In this technical solution, both the driving of the needle mounting member and the driving of the wing driving member are implemented by means of the operating member, and the operating member comprises the wing driving path, the transition path, and the needle driving path which are sequentially implemented. Hence, in the wing driving path, the operating member can only drive the wing driving member but cannot drive the needle mounting member. In this case, even if the operating member is incorrectly operated or accidentally touched, the operating member cannot drive the needle mounting member to move axially, and the suturing needles mounted on the needle mounting member thus cannot extend out of the tube body. In this way, accidental extension of the suturing needles can be completely avoided, thereby significantly improving the safety of using the tissue suturing device. By means of the transition path, the operating member is disengaged from the wing driving member and engaged with the needle mounting member. In this way, in the needle driving path, the operating member can drive the needle mounting member to move from the first position to the second position, such that the suturing needles extend out of the tube body.

In some embodiments, the operating member is driven to move axially along the wing driving path to the transition path, is rotated along the transition path by a preset angle and then reaches the needle driving path, and is driven to move axially along the needle driving path.

In some embodiments, when the operating member moves along the transition path to the needle driving path, the wing driving member maintained in the fourth position is capable of providing guidance for movement of the operating member along the needle driving path.

In a fourth aspect, the present invention provides a tissue suturing device capable of implementing the operating method of a tissue suturing device according to any of the foregoing described in the third aspect. In this way, as described above, the tissue suturing device can completely avoid accidental extension of the suturing needle, thereby significantly improving the safety of using the tissue suturing device.

It will be apparent that elements or features described in each of the above embodiments may be used individually or in combination in other embodiments.

### BRIEF DESCRIPTION OF DRAWINGS

In the drawings, the dimensions and proportions do not represent those of actual products. The drawings are illustrative only and certain non-essential elements or features are omitted for the sake of clarity.

FIG. 1 is a perspective view schematically showing a tissue suturing device according to an embodiment of the present invention from one angle of view, in which a half tube of a tube body is omitted to clearly show an operating assembly.

FIG. 2 is a partial schematic structural diagram of a half tube of the tissue suturing device in FIG. 1.

FIG. 3 is a perspective view of an operating member of the tissue suturing device in FIG. 1.

FIG. 4 is a schematic structural diagram in which an operating member, a wing driving member, and a needle mounting member of the tissue suturing device in FIG. 1 are in an initial position.

FIG. 5 is a perspective view of a needle mounting member of the tissue suturing device in FIG. 1 from one angle of view.

FIG. 6 is a perspective view of the needle mounting member of the tissue suturing device in FIG. 1 from another angle of view.

FIG. 7 is a perspective view of a positioning member of the tissue suturing device in FIG. 1.

FIG. 8 is a schematic structural diagram in which a positioning member and a tube body of the tissue suturing device in FIG. 1 are in a released state.

FIG. 9 is a perspective view of the tissue suturing device in FIG. 1 after completing a wing driving path, in which a half tube of a tube body is omitted to clearly show an operating assembly.

FIG. 10 is a schematic diagram in which the structure of FIG. 9 is partially enlarged.

FIG. 11 is a schematic structural diagram in which a positioning member and a tube body of the tissue suturing device in FIG. 9 are in a locking state.

FIG. 12 is a schematic structural diagram in which a mating section of a wing driving member is offset from a mating slot of an operating member in the tissue suturing device of FIG. 1 and FIG. 9.

FIG. 13 is a perspective view of the tissue suturing device in FIG. 9 after completing a transition path and a needle driving path, in which a half tube of a tube body is omitted to clearly show an operating assembly.

FIG. 14 is a schematic diagram in which the structure of FIG. 13 is partially enlarged.

FIG. 15 is a schematic structural diagram in which a mating section of a wing driving member extends into a mating slot of an operating member in the tissue suturing device of FIG. 13.

FIG. 16 is a schematic diagram schematically showing the alignment of a reference mark with a wing driving mark and a needle driving mark in an operating assembly of a tissue suturing device according to an embodiment of the present invention.

### DESCRIPTION OF REFERENCE SIGNS

1—tissue suturing device, 2—tube body, 211—half tube, 3—proximal end, 4—distal end, 5—axial channel, 6—needle mounting member, 7—wing driving member, 8—operating member, 9—wing driving guide slot, 10—transition guide slot, 11—needle driving guide slot, 12—guide column, 13—mating slot, 14—mating section, 15—axial through—hole, 16—axial open slot, 17—circumferential open slot, 18—extension section, 19—guide pin, 20—reference mark, 21—wing driving mark, 22—needle driving mark, 23—central through—hole, 24—positioning member, 25—operating opening, 26—locking opening, 27—elastic arm, 28—pressing part, 29—locking hook, 30—accommodating slot, 31—stop column, 32—stabilizing wing, 33—suturing needle, 34—mounting hole, 35—suturing needle channel, 36—axial recessed portion, and 37—connecting hole.

### DETAILED DESCRIPTION

The present invention will be described in detail below with reference to the accompanying drawings. The embodiments described herein are merely preferred embodiments according to the present invention, and on the basis of the preferred embodiments, those skilled in the art could conceive of other modes capable of implementing the present invention, which also fall within the scope of the present invention.

The proximal end mentioned in the present invention refers to an end of a tissue suturing device adjacent to or facing the operator when in use, and the distal end refers to an end of the tissue suturing device located away from or facing away from the operator when in use.

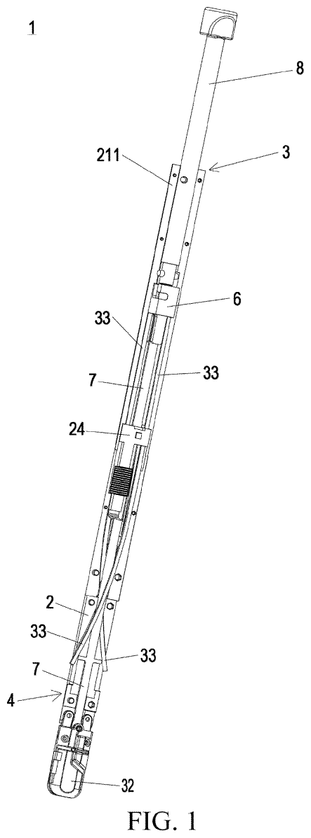

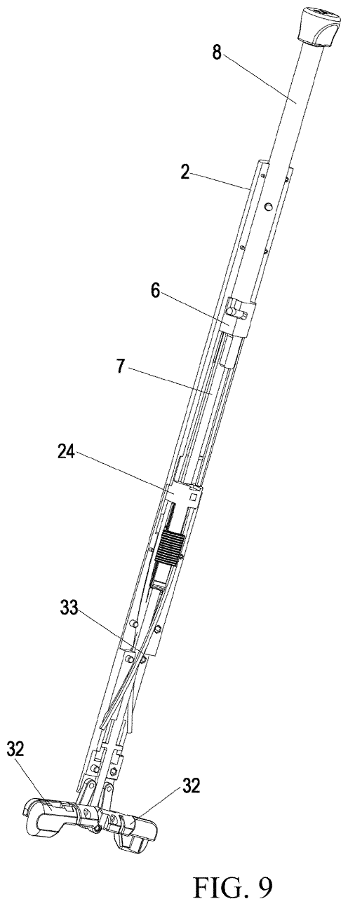

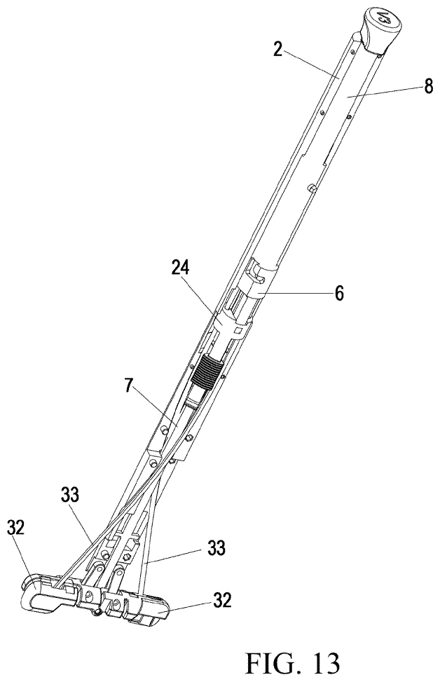

In a first aspect, with reference to FIG. 1, FIG. 9, and FIG. 13, the present invention provides an operating assembly for a tissue suturing device 1. The operating assembly includes a tube body 2, a needle mounting member 6, a wing driving member 7, and an operating member 8, where the tube body 2 includes a proximal end 3, a distal end 4, and an axial channel 5 extending between the proximal end 3 and the distal end 4. The proximal end 3 represents an end (such as the upper end in FIG. 1) of the tissue suturing device that is close to an operator during use, and the distal end 4 represents an end (such as the lower end in FIG. 1) of the tissue suturing device that is away from the operator during use. The needle mounting member 6 is axially and movably arranged in the axial channel. The wing driving member 7 is axially and movably arranged in the axial channel. At least a part of the operating member 8 is movably arranged in the axial channel. The operating member 8 includes a wing driving path, a transition path, and a needle driving path which are sequentially implemented. When moving along the wing driving path from an initial position to the transition path, the operating member 8 is not engaged with the needle mounting member 6 so that the needle mounting member 6 is allowed to be maintained in a first position, the first position being used to enable suturing needles of the tissue suturing device 1 to be in a retracted state or a retracted position. The operating member 8 is engaged with the wing driving member 7 so that the wing driving member 7 is driven to move towards the distal end to move from a third position used to enable stabilizing wings of the tissue suturing device 1 to be in a folded state or a folded position to a fourth position used to enable the stabilizing wings to be in an unfolded state or an unfolded position, and the wing driving member is capable of being maintained in the fourth position to maintain the stabilizing wings in the unfolded state or the unfolded position. When moving along the transition path to the needle driving path, the operating member 8 is disengaged from the wing driving member 7 and engaged with the needle mounting member 6; and when moving along the needle driving path, the operating member 8 drives the needle mounting member 6 to move from the first position to a second position used to enable the suturing needles to be in an extended state or a puncture position.

In this technical solution, both the driving of the needle mounting member 6 and the driving of the wing driving member 7 are implemented by means of the operating member 8, and the operating member 8 includes the wing driving path, the transition path, and the needle driving path which are sequentially implemented. Hence, during actual use, because the transition path is arranged between the wing driving path and the needle driving path, in the wing driving path, the operating member 8 can only drive the wing driving member 7 but cannot drive the needle mounting member 6. In this case, even if the operating member 8 is incorrectly operated or accidentally touched, the operating member 8 cannot drive the needle mounting member 6 to move axially, and the suturing needles 33 mounted on the needle mounting member 6 thus cannot extend out of the tube body 2. In this way, accidental extension of the suturing needles 33 can be completely avoided, thereby significantly improving the safety of using the tissue suturing device 1. By means of the transition path, the operating member 8 is disengaged from the wing driving member 7 and engaged with the needle mounting member 6. In this way, in the needle driving path, the operating member 8 can only drive the needle mounting member 6 to move from the first position to the second position, such that the suturing needles 33 extend out of the tube body 2. Therefore, the operating assembly provided by the present invention can sequentially operate the stabilizing wings and the suturing needles, and avoid accidental extension of the suturing needles, thereby significantly improving the safety of using the tissue suturing device.

In the operating assembly according to the present invention, the needle mounting member 6 can be maintained in the first position by means of a variety of structures. For example, in some embodiments, at least one protrusion is formed on the needle mounting member 6 and may be an elastic protrusion, a corresponding recess is formed on an inner surface of a sidewall of the tube body 2, and the protrusion can be cooperatively arranged in the recess. In this way, when the operating member 8 moves along the wing driving path from the initial position to the transition path, due to the fact that the operating member 8 is not engaged with the needle mounting member 6, the protrusion cannot be disengaged from the recess, such that the needle mounting member 6 is maintained in the first position. When the operating member 8 moves along the needle driving path, due to the fact that the operating member 8 is engaged with the needle mounting member 6, a driving force applied by the operating member 8 to the needle mounting member 6 drives the protrusion to be disengaged from the recess, such that the needle mounting member 6 is driven to move from the first position to the second position. In addition, reversely, the protrusion may be formed on the inner surface of the sidewall of the tube body 2, and the recess may be formed on the needle mounting member 6. For another example, in some embodiments, at least one elastic arm is formed on the needle mounting member 6, and a corresponding engagement platform is formed on the inner surface of the sidewall of the tube body 2. The elastic arm can be in engagement fit with the engagement platform to maintain the needle mounting member 6 in the first position. In this way, when the operating member 8 moves along the wing driving path from the initial position to the transition path, due to the fact that the operating member 8 is not engaged with the needle mounting member 6, the elastic arm and the engagement platform are maintained to be in engagement connection, such that the needle mounting member 6 is maintained in the first position. When the operating member 8 moves along the needle driving path, due to the fact that the operating member 8 is engaged with the needle mounting member 6, a driving force applied by the operating member 8 to the needle mounting member 6 drives the elastic arm to deform to be disengaged from the engagement platform, such that the needle mounting member 6 is driven to move from the first position to the second position.

In the operating assembly according to the present invention, in some embodiments, the wing driving path, the transition path, and the needle driving path are formed between the operating member 8 and the wing driving member 7 and the needle mounting member 6. In this case, the operating member 8 can move axially in all of the wing driving path, the transition path, and the needle driving path. That is, in the wing driving path, the operating member 8 moves axially by a first predetermined distance to drive the wing driving member 7 to move from the third position to the fourth position. In this case, the wing driving member is maintained in the fourth position, and the operating member 8 is not engaged with the needle mounting member 6 (that is, the needle mounting member 6 is not driven by the operating member 8) so that the needle mounting member 6 is allowed to be maintained in the first position. In the transition path, the operating member 8 continues to move axially by a second predetermined distance, such that the operating member 8 is disengaged from the wing driving member 7 and engaged with the needle mounting member 6. In the needle driving path, the operating member 8 continues to move axially by a third predetermined distance, such that the needle mounting member 6 is driven to move from the first position to the second position.

In the operating assembly according to the present invention, in some embodiments, the wing driving path, the transition path, and the needle driving path are formed between the operating member 8 and the wing driving member 7 and the needle mounting member 6. In this case, the operating member 8 can be rotated in all of the wing driving path, the transition path, and the needle driving path. For example, in the wing driving path, the operating member 8 is rotated by a first predetermined angle, such that the wing driving member 7 is driven to move from the third position to the fourth position by means of a cam or a tapered profile between the operating member 8 and the wing driving member 7. In this case, the wing driving member is maintained in the fourth position, while the operating member 8 and the needle mounting member 6 (such as by means of mating between a circumferential sliding slot and a column between the two) are maintained to be in relative rotation for disengagement, such that the needle mounting member 6 is maintained in the first position. In the transition path, the operating member 8 continues to rotate by a second predetermined angle, such that the operating member 8 and the wing driving member 7 (such as by means of mating between a circumferential sliding slot and a column between the two) are maintained to be in relative rotation for disengagement, and the operating member 8 is engaged with the needle mounting member 6. In the needle driving path, the operating member 8 continues to rotate by a third predetermined angle, such that the needle mounting member 6 is driven to move from the first position to the second position by means of a cam or a tapered profile between the operating member 8 and the needle mounting member 6.

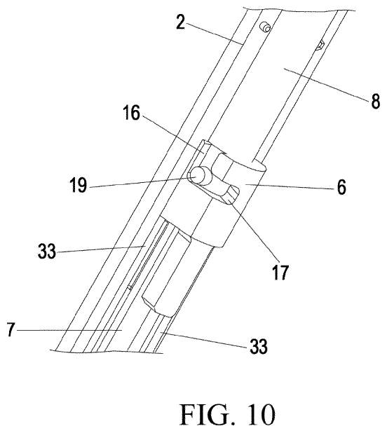

In the operating assembly according to the present invention, in some embodiments, with reference to FIG. 1, FIG. 9, and FIG. 13, the wing driving path, the transition path, and the needle driving path are formed between the tube body 2 and the operating member 8, where the wing driving path extends toward the proximal end 3 and along the axial direction, the needle driving path extends toward the distal end 4 and along the axial direction, and the transition path is arranged along a circumferential direction of the tube body 2 (to enable the operating member 8 to rotate by a preset angle; for example, in FIG. 10, the operating member 8 rotates anticlockwise by a preset angle), such that the operating member 8 can move axially along the wing driving path, then rotate along the transition path by a preset angle and then reach the needle driving path, and then move axially along the needle driving path. For example, during extension of the needles, the operating member 8 first performs a first axial movement to complete the wing driving path, then performs one rotation to complete the transition path, and finally performs one axial movement to complete the needle driving path. In this way, the transition path separates the wing driving path from the needle driving path, and the operating member 8 cannot implement the needle driving path when implementing the wing driving path, thereby completely avoiding accidental extension of the suturing needles. In addition, the transition path may be arranged along the circumferential direction of the tube body 2 by forming a certain inclination angle, such as 1-2°, preferably 1.5°. By forming a certain inclination angle, it is easier for the operating member to rotate by a certain preset angle when moving along the transition path.

In addition, the preset angle may be a desired angle, such as 30°, 45°, or 60°. Certainly, the preset angle may also be other angles.

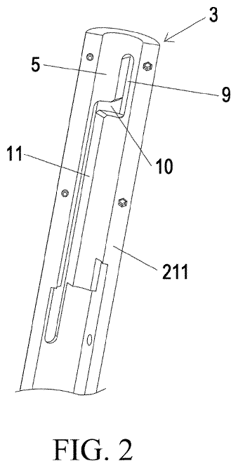

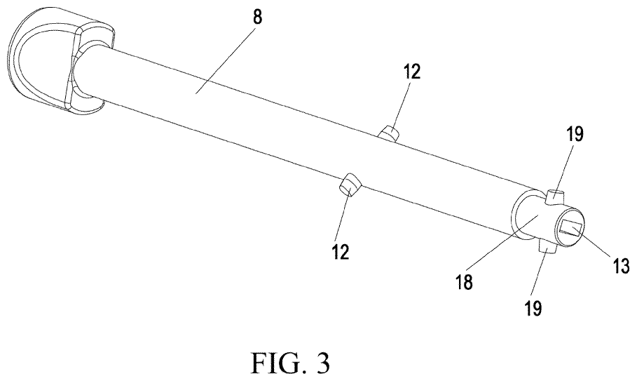

The wing driving path, the transition path, and the needle driving path between the tube body 2 and the operating member 8 may be implemented in various manners. For example, in some embodiments, with reference to FIG. 2 and FIG. 3, a wing driving guide slot 9 providing the wing driving path, a transition guide slot 10 providing the transition path, and a needle driving guide slot 11 providing the needle driving path are formed on an axial sidewall of the tube body 2, where a guide column 12 is provided on the operating member 8 and is movable along the wing driving guide slot 9, the transition guide slot 10, and the needle driving guide slot 11. In this way, when moving along the wing driving guide slot 9, the guide column 12 will not be able to move in the needle driving guide slot 11. Therefore, the operating member 8 can only implement the wing driving path but cannot implement the needle driving path. In this way, only the stabilizing wings can be unfolded but the suturing needles are unable to extend out, thereby completely avoiding accidental extension of the suturing needles. Only when the guide column 12 moves along the transition guide slot 10 to the needle driving guide slot 11 (that is, rotating by a preset angle), the further axial movement of the operating member 8 can drive the needle mounting member 6 to move axially so that the suturing needles extend out of the tube body 2. In addition, the wing driving guide slot 9, the transition guide slot 10, and the needle driving guide slot 11 may be recesses formed on the inner surface of the sidewall of the tube body 2, or the wing driving guide slot 9, the transition guide slot 10, and the needle driving guide slot 11 may be through-channels formed on the sidewall of the tube body 2 and penetrating the thickness of the sidewall. In addition, in an alternative embodiment, the wing driving guide slot 9, the transition guide slot 10, and the needle driving guide slot 11 may be formed on the operating member 8, and the guide column 12 may be formed on the inner surface of the sidewall of the tube body 2.

In addition, in some embodiments, in order to improve the convenience of installation and easy processing of the operating assembly, with reference to FIG. 1 and FIG. 2, the tube body 2 includes two half tubes 211 capable of being joined together, where the wing driving path, the transition path, and the needle driving path are formed between at least one of the two half tubes 211 and the operating member 8. In this way, the wing driving path, the transition path, and the needle driving path can be easily formed between at least one half tube 211 and the operating member 8. During assembly, the two half tubes 211 can also be easily assembled into the tube body 2.

In addition, in some embodiments, in order to improve the operational stability and reliability of the operating member 8, with reference to FIG. 2 and FIG. 3, each half tube 211 is formed with a wing driving guide slot 9, a transition guide slot 10, and a needle driving guide slot 11, two guide columns 12 are provided on the operating member 8, and each guide column 12 is movable along the wing driving guide slot 9, the transition guide slot 10, and the needle driving guide slot 11 of a half tube corresponding thereto. For example, in some embodiments, each guide column 12 can move axially along the wing driving guide slot 9 of the half tube corresponding thereto to unfold the stabilizing wings, then rotate along the transition guide slot 10 corresponding thereto by a preset angle to reach the needle driving guide slot 11, and then move axially along the needle driving guide slot 11 corresponding thereto to extend the suturing needles to the puncture position. In this way, each set of the wing driving guide slot 9, the transition guide slot 10, and the needle driving guide slot 11 is matched with each guide column 12, such that the operational stability of the operating member 8 can be significantly improved.

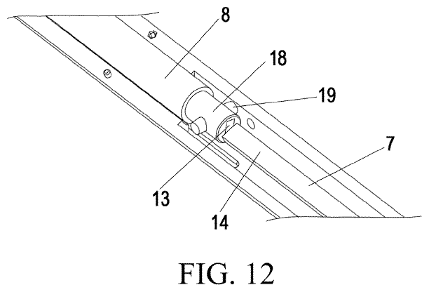

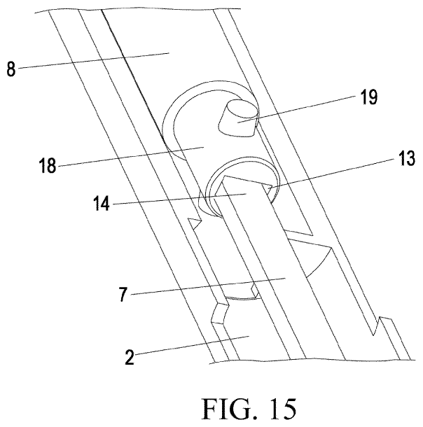

In addition, in some embodiments, in order to further improve the stability of extending the suturing needles, when the operating member 8 moves along the transition path to the needle driving path, the wing driving member 7 maintained in the fourth position can provide guidance for movement (such as axial movement) of the operating member 8 along the needle driving path. In this way, when the operating member 8 moves along the needle driving path, the wing driving member 7 provides guidance for the operating member 8, which can further improve the stability of the operating member 8 when moving along the needle driving path. For example, in the examples of FIG. 12 and FIG. 15, the wing driving member 7 maintained in the fourth position can provide guidance for axial movement of the operating member 8 along the needle driving path, so as to stably guide the axial movement of the operating member 8.

In some embodiments, with reference to FIG. 12 and FIG. 15, an axially extending mating slot 13 is formed on an end surface of one of the operating member 8 and the wing driving member 7, and an axially extending mating section 14 is formed at an end part of the other of the operating member and the wing driving member. When the operating member 8 moves axially along the wing driving path, the mating section 14 is offset from the mating slot 13, such that the operating member 8 is engaged with the wing driving member 7 to drive the wing driving member 7 to move axially from the third position to the fourth position; when the operating member 8 rotates along the transition path, the mating section 14 is gradually axially aligned with the mating slot 13; when the operating member 8 rotates along the transition path to the needle driving path, the mating section 14 is axially aligned with the mating slot 13, such that the operating member 8 is disengaged from the wing driving member 7; and when the operating member 8 moves axially along the needle driving path, the mating section 14 enters the mating slot 13 to provide guidance. At the time, due to the fact that the wing driving member 7 is maintained in the fourth position, when the mating section 14 enters the mating slot 13, the wing driving member 7 is not driven by the operating member 8.

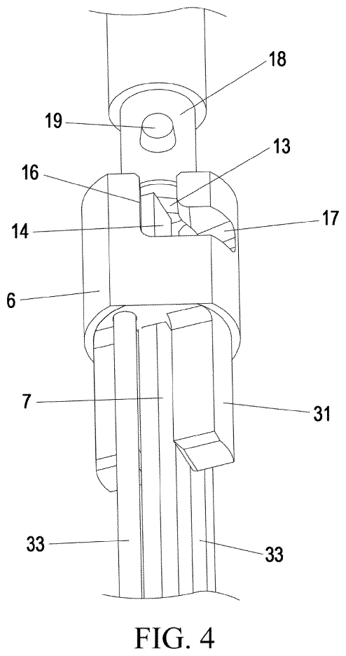

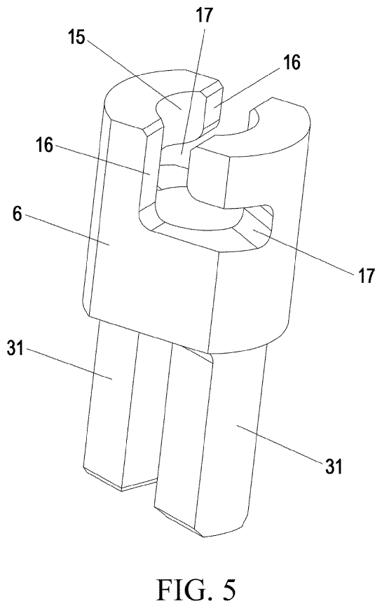

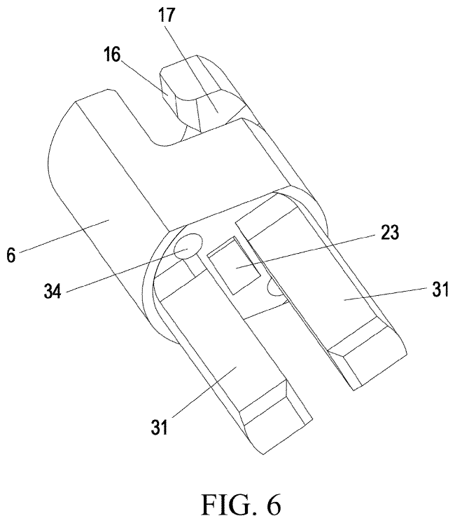

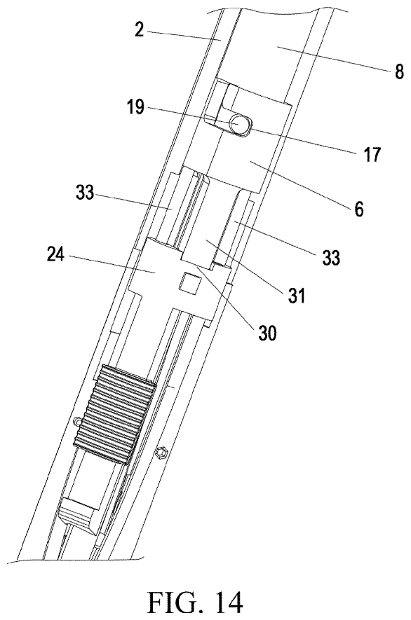

In addition, in the operating assembly according to the present invention, the needle mounting member 6 may have a variety of structural forms. No matter which structural form is used for the needle mounting member 6, said structural form is acceptable as long as the above function can be implemented. For example, in some embodiments, with reference to FIG. 4, FIG. 5, and FIG. 6, the needle mounting member 6 is formed with an axial through-hole 15, an axial open slot 16 and a circumferential open slot 17 communicating with each other are formed on a sidewall of the axial through-hole 15, an extension section 18 capable of extending into the axial through-hole 15 is formed at a distal end of the operating member 8, and a guide pin 19 radially extending outwards is arranged on the extension section 18. With reference to FIG. 4 and FIG. 10, the operating member 8, when moving axially along the wing driving path towards the distal end, drives the guide pin 19 to slide axially along the axial open slot 16, such that the operating member 8 does not drive the needle mounting member 6 to move axially towards the distal end, enabling the needle mounting member 6 to be maintained in the first position. With reference to FIG. 10 and FIG. 14, the operating member 8, when rotating circumferentially along the transition path, drives the guide pin 19 to slide circumferentially along the circumferential open slot 17, such that the operating member 8 is engaged with the needle mounting member 6, thereby driving the needle mounting member 6 to move axially.

In addition, in some embodiments, there may be one axial open slot 16 and one circumferential open slot 17, and correspondingly, there may be one guide pin 19. In addition, in some embodiments, with reference to FIG. 3, FIG. 4, and FIG. 5, at least two sets of the axial open slot 16 and the circumferential open slot 17 are formed on the sidewall of the axial through-hole 15, and at least two guide pins 19 are provided on the extension section 18, where each guide pin 19 is capable of sliding along a set of the axial open slot 16 and the circumferential open slot 17 corresponding thereto. In this way, by means of the at least two guide pins 19 and the at least two sets of the axial open slot 16 and the circumferential open slot 17, the stability of driving the needle mounting member 6 by the operating member 8 can be further improved.

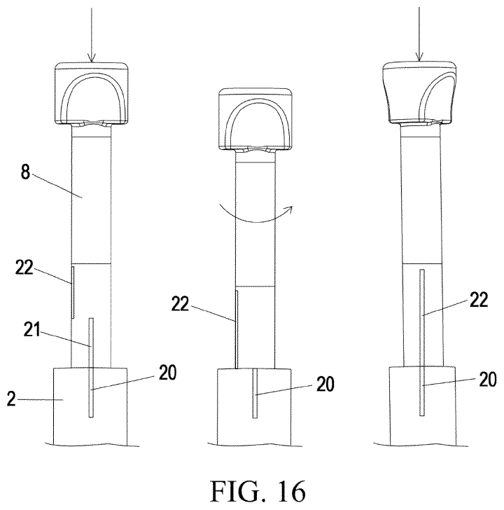

In addition, in some embodiments, in order to improve the operability of the operating assembly and further enable the operator to quickly identify the wing driving path, the transition path, and the needle driving path of the operating member 8, with reference to FIG. 16, a reference mark 20 is arranged on the tube body 2, a wing driving mark 21 and a needle driving mark 22 circumferentially spaced apart at a preset angle and axially spaced apart from each other are arranged on the operating member 8, and the wing driving mark 21 is different from the needle driving mark 22. When the wing driving mark 21 is axially aligned with the reference mark 20, the operating member 8 is capable of moving axially along the wing driving path from the initial position, such that the operator can quickly identify the wing driving path of the operating member 8. When the operating member 8 rotates along the transition path by a preset angle (the preset angle being the angle of the circumferential rotation) and reaches the needle driving path, the needle driving mark 22 is axially aligned with the reference mark 20, such that the operator can quickly identify the needle driving path of the operating member 8. In addition, in order to further improve identifiability, the wing driving mark 21 and the needle driving mark 22 may have different colors. For example, the wing driving mark 21 is blue, the needle driving mark 22 is red, and the reference mark 20 may be red.

In addition, in some embodiments, in order to improve the stability of movement of the wing driving member 7 and the needle operating member 6, the wing driving member 7 and the needle operating member 6 can provide guidance for each other. For example, in some embodiments, the needle mounting member 6 is formed with a central through-hole 23, and the wing driving member 7 passes through the central through-hole 23. Moreover, such a cooperative relationship can further improve the structural compactness of the operating assembly.

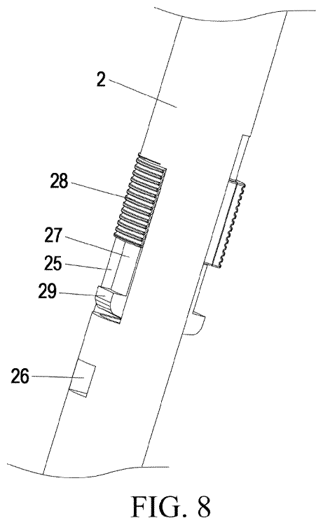

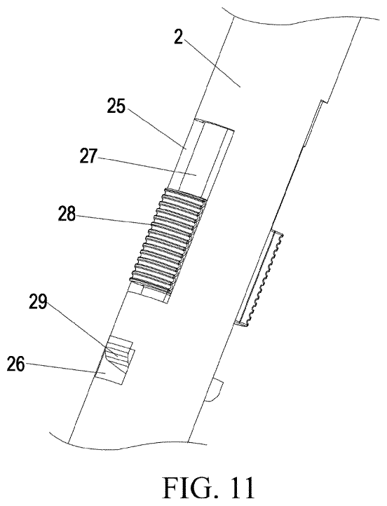

In addition, in the operating assembly according to the present invention, the wing driving member 7 can be maintained in the fourth position by various methods. For example, in one method, an elastic protrusion is arranged on an inner side surface of the tube body 2, and a recess is formed on the wing driving member 7. When the wing driving member 7 moves to the fourth position, the elastic protrusion enters the recess, thereby maintaining the wing driving member 7 in the fourth position. For another example, in another method, with reference to FIG. 1, FIG. 8, FIG. 9, and FIG. 11, the wing driving member 7 is provided with a positioning member 24, and a releasable locking structure is formed between the positioning member 24 and the tube body 2. When the operating member 8 moves along the wing driving path from the initial position to the transition path, the locking structure is in a locking state to maintain the wing driving member 7 in the fourth position, such that the wing driving member 7 can be effectively prevented from moving accidentally to accidentally fold the unfolded stabilizing wings. When the locking structure is released, the wing driving member 7 is allowed to move from the fourth position to the third position. In addition, when the wing driving member 7 drives the positioning member 24 to move axially in the tube body 2, there is a guiding effect between the positioning member 24 and the tube body 2. Such a guiding effect can further improve the stability of the axial movement of the wing driving member 7 and drive the stabilizing wings to be unfolded more stably. In addition, with reference to FIG. 1, the positioning member 24 may be positioned in the approximately middle part of the wing driving member 7, thereby providing a support point to the wing driving member 7 in the approximately middle part of the wing driving member 7. Such a support point is particularly advantageous when the wing driving member 7 is an axially extending long rod. Certainly, the positioning member 24 may also be arranged at other positions on the wing driving member 7.

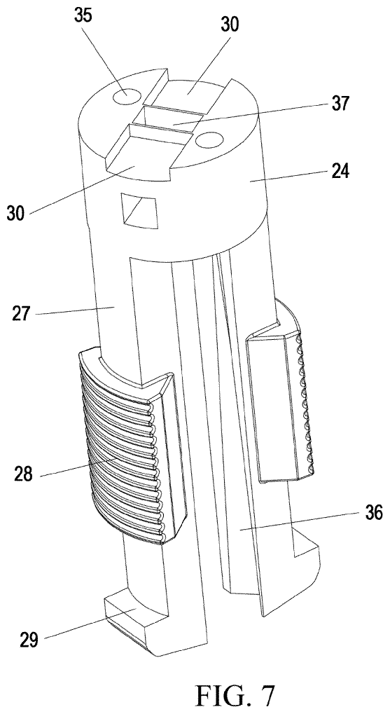

In addition, in the operating assembly according to the present invention, the positioning member 24 and the locking structure may have a variety of structural forms. No matter which structural form is used, said structural form is acceptable as long as the above effect can be implemented. For example, in some embodiments, with reference to FIG. 7, FIG. 8, and FIG. 11, an operating opening 25 and a locking opening 26 axially spaced apart from each other are formed on a sidewall of the tube body 2, the positioning member 24 includes an elastic arm 27, the elastic arm 27 is provided with a pressing part 28 and a locking hook 29 axially spaced apart from each other, and the pressing part 28 protrudes outwards radially from the operating opening 25. The locking structure includes the locking opening 26 and the locking hook 29, and is in the locking state when the locking hook 29 is in the locking opening 26. The elastic arm 27 is pressed by means of the pressing part 28, such that the locking structure is released when the locking hook 29 is disengaged from the locking opening 26. In this way, in the initial position, the locking hook 29 is located in the operating opening 25. When the wing driving member 7 moves from the third position to the fourth position, a front end of the locking hook 29 is pressed by a distal wall of the operating opening 25 to cause elastic deformation of the elastic arm 27 and to retract the locking hook 29 back into the tube body 2. With reference to FIG. 11, when the wing driving member 7 moves to the fourth position, due to the restoration of the elastic arm 27, the locking hook 29 protrudes from the locking opening 26 to be in a locking state, so as to maintain the wing driving member 7 in the fourth position. In addition, the pressing part 28 of the elastic arm 27 is pressed to cause the locking hook 29 to leave the locking opening 26 and retract back into the tube body 2 for release, such that the wing driving member 7 is allowed to move from the fourth position to the third position.

In addition, in some embodiments, with reference to FIG. 7, the positioning member 24 is formed with suturing needle channels 35 used for allowing the suturing needles to pass through axially. In this way, the suturing needle channels 35 can provide support and guidance for the slender suturing needles, enabling the suturing needles to extend more stably. In addition, in some embodiments, with reference to FIG. 7, axial recessed portions 36 allowing the suturing needles to pass through are formed on the elastic arm 27 of the positioning member 24. In this way, the axial recessed portions 36 can accommodate the suturing needles and provide support for the suturing needles, enabling the suturing needles to move more stably.

In addition, in order to further improve the accuracy of the suturing needles moving to the second position, in some embodiments, with reference to FIG. 13, when the wing driving member 7 is maintained in the fourth position, the positioning member 24 is in a locking state. In this case, when the needle mounting member 6 is in the second position, the positioning member 24 stops the needle mounting member 6. In this way, the further axial movement of the needle mounting member 6 can be prevented, such that the needle mounting member 6 can stably and reliably stop in the second position.

In addition, the positioning member 24 can stop the needle mounting member 6 in various forms. For example, in one form, a proximal end surface of the positioning member 24 is in direct contact with a distal end surface of the needle mounting member 6. In another form, with reference to FIG. 7 and FIG. 14, an accommodating slot 30 is formed on a proximal end surface of the positioning member 24, a stop column 31 is formed at a distal end of the needle mounting member 6, and when the needle mounting member 6 is in the second position, the stop column 31 extends into the accommodating slot 30. In this case, because the stop column 31 extends into the accommodating slot 30, the needle mounting member 6 can be prevented from shaking, making the suturing needles more stable.

In addition, with reference to FIG. 7, a connecting hole 37 is formed on the proximal end surface of the positioning member 24. The wing driving member 7 can be allowed to pass through the connecting hole 37. A locking pin shaft (not shown in the figure) can radially pass through the positioning member 24 and the wing driving member 7, such that the positioning member 24 is disposed at the wing driving member 7 at a position limited by the locking pin shaft. In addition, the connecting hole 37 may be located between two accommodating slots 30.

In a second aspect, the present invention provides a tissue suturing device 1. With reference to FIG. 1, FIG. 9, and FIG. 13, the tissue suturing device 1 includes the operating assembly according to any of the foregoing described in the first aspect above, stabilizing wings 32, and suturing needles 33. The stabilizing wings 32 are hinged to the distal end 4 of the tube body 2 and hinged to the wing driving member 7, and the suturing needles 33 are arranged on the needle mounting member 6 and located in the tube body 2. The stabilizing wings 32 are capable of being unfolded and folded, and the suturing needles 33 are capable of extending out of the tube body 2 to a puncture position and retracting back into the tube body 2. In this way, as described in the first aspect above, by means of the operating assembly, the tissue suturing device can completely avoid accidental extension of the suturing needle, thereby significantly improving the safety of using the tissue suturing device.

In a third aspect, the present invention provides an operating method of a tissue suturing device. The operating method includes a wing driving path, a transition path, and a needle driving path which are sequentially implemented, where an operating member of a tissue suturing device is driven to move along the wing driving path from an initial position to the transition path, such that the operating member only drives a wing driving member of the tissue suturing device to move from a third position to a fourth position and be maintained in the fourth position, so as to unfold stabilizing wings of the tissue suturing device; the operating member is driven to move along the transition path to the needle driving path, such that the operating member is disengaged from the wing driving member and engaged with a needle mounting member of the tissue suturing device; and the operating member is driven to move along the needle driving path to drive the needle mounting member to move from a first position to a second position, such that suturing needles are driven to extend out of a tube body of the tissue suturing device to a puncture position.

In the operating method of a tissue suturing device, both the driving of the needle mounting member and the driving of the wing driving member are implemented by means of the operating member, and the operating member includes the wing driving path, the transition path, and the needle driving path which are sequentially implemented. Hence, in the wing driving path, the operating member can only drive the wing driving member but cannot drive the needle mounting member. In this case, even if the operating member is incorrectly operated or accidentally touched, the operating member cannot drive the needle mounting member to move axially, and the suturing needles mounted on the needle mounting member thus cannot extend out of the tube body. In this way, accidental extension of the suturing needles can be completely avoided, thereby significantly improving the safety of using the tissue suturing device. By means of the transition path, the operating member is disengaged from the wing driving member and engaged with the needle mounting member. In this way, in the needle driving path, the operating member can drive the needle mounting member to move from the first position to the second position, such that the suturing needles extend out of the tube body. When the stabilizing wings and the suturing needles need to be folded and retracted, only the reverse operation is required.

In addition, in some embodiments, the operating member is driven to move axially along the wing driving path to the transition path, is rotated along the transition path by a preset angle and then reaches the needle driving path, and is driven to move axially along the needle driving path. During extension of the needles, the operating member first performs a first axial movement to complete the wing driving path, then performs one rotation to complete the transition path, and finally performs one axial movement to complete the needle driving path. In this way, the transition path separates the wing driving path and the needle driving path, and the operating member cannot implement the needle driving path when implementing the wing driving path, thereby completely avoiding accidental extension of the suturing needle. In addition, such an arrangement enables an operator to effectively identify each driving path.

In addition, in some embodiments, when the operating member moves along the transition path to the needle driving path, the wing driving member maintained in the fourth position is capable of providing guidance for movement (such as axial movement) of the operating member along the needle driving path. In this way, when the operating member moves along the needle driving path, the wing driving member provides the guidance for the operating member, which can further improve the stability of the operating member when moving along the needle driving path. For example, in the examples of FIG. 12 and FIG. 15, the wing driving member maintained in the fourth position can provide guidance for axial movement of the operating member along the needle driving path, so as to stably guide the axial movement of the operating member.

In a fourth aspect, the present invention provides a tissue suturing device capable of implementing the operating method of a tissue suturing device according to any of the foregoing described in the third aspect above. In this way, as described above, the tissue suturing device can completely avoid accidental extension of the suturing needle, thereby significantly improving the safety of using the tissue suturing device.

The scope of protection of the present invention is defined only by the claims. In light of the teachings of the present invention, those skilled in the art would readily recognize that alternative structures of the structures disclosed in the present invention may be used as feasible alternative embodiments, and the embodiments disclosed in the present invention may be combined to form new embodiments, which also fall within the scope of the appended claims.

1. An operating assembly for a tissue suturing device (1), the operating assembly comprising:
a tube body (2), the tube body (2) comprising a proximal end (3), a distal end (4), and an axial channel (5) extending between the proximal end (3) and the distal end (4);
a needle mounting member (6), the needle mounting member (6) being axially and movably arranged in the axial channel;
a wing driving member (7), the wing driving member (7) being axially and movably arranged in the axial channel; and
an operating member (8), at least a part of the operating member (8) being movably arranged in the axial channel;
the operating member (8) comprising a wing driving path, a transition path, and a needle driving path which are sequentially implemented, wherein:
when moving along the wing driving path from an initial position to the transition path, the operating member (8) is not engaged with the needle mounting member (6) so that the needle mounting member (6) is allowed to be maintained in a first position used to enable suturing needles to be in a retracted state, the operating member (8) is engaged with the wing driving member (7) so that the wing driving member (7) is driven to move from a third position to a fourth position, the third position is used to enable stabilizing wings to be in a folded state, the fourth position is used to enable the stabilizing wings to be in an unfolded state, and the wing driving member is capable of being maintained in the fourth position;
when moving along the transition path to the needle driving path, the operating member (8) is disengaged from the wing driving member (7) and engaged with the needle mounting member (6); and
when moving along the needle driving path, the operating member (8) drives the needle mounting member (6) to move from the first position to a second position used to enable the suturing needles to be in an extended state.

2. The operating assembly according to claim 1, wherein the wing driving path, the transition path, and the needle driving path are formed between the tube body (2) and the operating member (8), wherein the wing driving path extends toward the proximal end (3) and along the axial direction, the needle driving path extends toward the distal end (4) and along the axial direction, and the transition path is arranged along a circumferential direction of the tube body (2), such that the operating member (8) is capable of moving axially along the wing driving path, then rotating along the transition path by a preset angle and then reaching the needle driving path, and then moving axially along the needle driving path.

3. The operating assembly according to claim 2, wherein a wing driving guide slot (9) providing the wing driving path, a transition guide slot (10) providing the transition path, and a needle driving guide slot (11) providing the needle driving path are formed on an axial sidewall of the tube body (2), wherein a guide column (12) is provided on the operating member (8), and the guide column (12) is movable along the wing driving guide slot (9), the transition guide slot (10), and the needle driving guide slot (11).

4. The operating assembly according to claim 2, wherein the tube body (2) comprises two half tubes (211) capable of being joined together, wherein the wing driving path, the transition path, and the needle driving path are formed between at least one of the two half tubes (211) and the operating member (8).

5. The operating assembly according to claim 4, wherein each of the half tubes (211) is formed with a wing driving guide slot (9), a transition guide slot (10), and a needle driving guide slot (11), two guide columns (12) are provided on the operating member (8), and each of the guide columns (12) is movable along the wing driving guide slot (9), the transition guide slot (10), and the needle driving guide slot (11) of a half tube corresponding thereto.

6. The operating assembly according to claim 1, wherein when the operating member (8) moves along the transition path to the needle driving path, the wing driving member (7) maintained in the fourth position is capable of providing guidance for movement of the operating member (8) along the needle driving path.

7. The operating assembly according to claim 6, wherein an axially extending mating slot (13) is formed on an end surface of one of the operating member (8) and the wing driving member (7), and an axially extending mating section (14) is formed at an end part of the other of the operating member and the wing driving member, wherein:
when the operating member (8) moves axially along the wing driving path, the mating section (14) is offset from the mating slot (13), such that the operating member (8) is engaged with the wing driving member (7);
when the operating member (8) rotates along the transition path to the needle driving path, the mating section (14) is axially aligned with the mating slot (13), such that the operating member (8) is disengaged from the wing driving member (7); and
when the operating member (8) moves axially along the needle driving path, the mating section (14) enters the mating slot (13) to provide the guidance.

8. The operating assembly according to claim 2, wherein:
the needle mounting member (6) is formed with an axial through-hole (15), and an axial open slot (16) and a circumferential open slot (17) communicating with each other are formed on a sidewall of the axial through-hole (15);
an extension section (18) capable of extending into the axial through-hole (15) is formed at a distal end of the operating member (8), and a guide pin (19) radially extending outwards is arranged on the extension section (18); and
the operating member (8), when moving axially along the wing driving path towards the distal end, drives the guide pin (19) to slide axially along the axial open slot (16), such that the operating member (8), when not driving the needle mounting member (6) to move axially towards the distal end, enables the needle mounting member (6) to be maintained in the first position; and the operating member (8), when rotating circumferentially along the transition path, drives the guide pin (19) to slide circumferentially along the circumferential open slot (17), such that the operating member (8) is engaged with the needle mounting member (6).

9. The operating assembly according to claim 8, wherein at least two sets of the axial open slot (16) and the circumferential open slot (17) are formed on the sidewall of the axial through-hole (15), and at least two guide pins (19) are provided on the extension section (18), wherein each of the guide pins (19) is capable of sliding along a set of the axial open slot (16) and the circumferential open slot (17) corresponding thereto.

10. The operating assembly according to claim 2, wherein a reference mark (20) is arranged on the tube body (2), a wing driving mark (21) and a needle driving mark (22) which are circumferentially spaced apart at a preset angle and axially spaced apart from each other are arranged on the operating member (8), and the wing driving mark (21) is different from the needle driving mark (22), wherein:
when the wing driving mark (21) is axially aligned with the reference mark (20), the operating member (8) is capable of moving axially along the wing driving path from the initial position; and
when the operating member (8) rotates along the transition path by the preset angle to reach the needle driving path, the needle driving mark (22) is axially aligned with the reference mark (20).

11. The operating assembly according to claim 1, wherein the needle mounting member (6) is formed with a central through-hole (23), and the wing driving member (7) passes through the central through-hole (23).

12. The operating assembly according to claim 1, wherein:
the wing driving member (7) is provided with a positioning member (24), and a releasable locking structure is formed between the positioning member (24) and the tube body (2), wherein when the operating member (8) moves along the wing driving path from the initial position to the transition path, the locking structure is in a locking state to maintain the wing driving member (7) in the fourth position; and
when the locking structure is released, the wing driving member (7) is allowed to move from the fourth position to the third position.

13. The operating assembly according to claim 12, wherein:
an operating opening (25) and a locking opening (26) axially spaced apart from each other are formed on a sidewall of the tube body (2), the positioning member (24) comprises an elastic arm (27), the elastic arm (27) is provided with a pressing part (28) and a locking hook (29) axially spaced apart from each other, and the pressing part (28) protrudes outwards radially from the operating opening (25); and
the locking structure comprises the locking opening (26) and the locking hook (29), and the locking structure is in the locking state when the locking hook (29) is in the locking opening (26); and the elastic arm (27) is pressed by means of the pressing part (28), such that the locking structure is released when the locking hook (29) is disengaged from the locking opening (26).

14. The operating assembly according to claim 12, wherein the suturing needles are allowed to pass through one or both of:
suturing needle channels (35) formed in the positioning member (24), or
axial recessed portions (36) formed on the elastic arm (27) of the positioning member (24).

15. The operating assembly according to claim 12, wherein when the needle mounting member (6) is in the second position, the positioning member (24) stops the needle mounting member (6).

16. The operating assembly according to claim 15, wherein an accommodating slot (30) is formed on a proximal end surface of the positioning member (24), a stop column (31) is formed at a distal end of the needle mounting member (6), and when the needle mounting member (6) is in the second position, the stop column (31) extends into the accommodating slot (30).

17. A tissue suturing device, comprising:
the operating assembly according to claim 1;
stabilizing wings (32), the stabilizing wings (32) being hinged to the distal end (4) of the tube body (2) and hinged to the wing driving member (7); and
suturing needles (33), the suturing needles (33) being arranged on the needle mounting member (6) and located in the tube body (2);
wherein the stabilizing wings (32) are capable of being unfolded and folded, and the suturing needles (33) are capable of extending out of the tube body (2) to a puncture position and retracting back into the tube body (2).

18. An operating method of a tissue suturing device, the operating method comprising a wing driving path, a transition path, and a needle driving path which are sequentially implemented, wherein:
an operating member of a tissue suturing device is driven to move along the wing driving path from an initial position to the transition path, such that the operating member only drives a wing driving member of the tissue suturing device to move from a third position to a fourth position and be maintained in the fourth position, so as to unfold stabilizing wings of the tissue suturing device;
the operating member is driven to move along the transition path to the needle driving path, such that the operating member is disengaged from the wing driving member and engaged with a needle mounting member of the tissue suturing device; and
the operating member is driven to move along the needle driving path to drive the needle mounting member to move from a first position to a second position, such that suturing needles are driven to extend out of a tube body of the tissue suturing device to a puncture position.

19. The operating method of a tissue suturing device according to claim 18, wherein the operating member is driven to move axially along the wing driving path to the transition path, is rotated along the transition path by a preset angle and then reaches the needle driving path, and is driven to move axially along the needle driving path.

20. The operating method of a tissue suturing device according to claim 18, wherein when the operating member moves along the transition path to the needle driving path, the wing driving member maintained in the fourth position provides guidance for movement of the operating member along the needle driving path.

21. (canceled)
